(19) United States
(12) Patent Application Publication
(10) Pub. No.: US 2023/0320640 A1
(43) Pub. Date: Oct. 12, 2023

(54) BIDIRECTIONAL SIGHTLINE-POSITION DETERMINATION DEVICE, BIDIRECTIONAL SIGHTLINE-POSITION DETERMINATION METHOD, AND TRAINING METHOD

(71) Applicants: NATIONAL UNIVERSITY CORPORATION CHIBA UNIVERSITY, Chiba-shi (JP); Sumitomo Pharma Co., Ltd., Osaka-shi (JP)

(72) Inventors: Toshiya NAKAGUCHI, Chiba-shi (JP); Kyohei FUKUDA, Chiba-shi (JP); Eiji SHIMIZU, Chiba-shi (JP); Yoshiyuki HIRANO, Chiba-shi (JP); Naomi SHIRAIWA, Suita-shi (JP); Yuki IKEDA, Suita-shi (JP); Masanori MIYAUCHI, Osaka-shi (JP)

(73) Assignees: NATIONAL UNIVERSITY CORPORATION CHIBA UNIVERSITY, Chiba-shi (JP); Sumitomo Pharma Co., Ltd., Osaka-shi (JP)

(21) Appl. No.: 18/192,827

(22) Filed: Mar. 30, 2023

(30) Foreign Application Priority Data
Apr. 6, 2022 (JP) 2022-063638

(51) Int. Cl. A61B 5/16; G06T 7/70; G06T 7/00

(52) U.S. Cl. CPC A61B 5/163; G06T 7/70; G06T 7/0012; G06T 2207/30201; G06T 2207/30004

(57) ABSTRACT

A determination device includes an acquisition unit, a first determination unit, a second determination unit, and an output unit. The acquisition unit is configured to acquire a sightline direction of a user and a sightline direction of at least one facing person facing the user. The first determination unit is configured to determine whether a sightline of the user is directed to the facing person based on the sightline direction of the user acquired by the acquisition unit. The second determination unit is configured to determine whether a sightline of the facing person is directed to the user based on the sightline direction of the facing person acquired by the acquisition unit. The output unit is configured to output a feedback to the user and/or an executor based on a result of a determination of the first determination unit and a result of a determination of the second determination unit.

### TECHNICAL FIELD

The present disclosure relates to a determination device, a determination method, a training method, and a program.

### DESCRIPTION OF THE RELATED ART

Currently, as one of effective options of treatment for neuropsychiatric disorders, for example, there has been executed Cognitive behavioral therapy as disclosed in NPL 1.

### PATENT DOCUMENT

### Non Patent Literature

Non Patent Literature 1: Yoshinaga et al., Psychotherapy and Psychosomatics, 2016, 85(4), p. 208-217.

### SUMMARY

### Technical Problem

There are not enough medical executors who can practice the therapy disclosed in Non-Patent Literature 1, and therefore, this method is not available to everyone who wishes to receive the therapy.

An object of the present disclosure is to provide a determination device, a determination method, a training method, and a program, which are capable of reproducing a sightline communication environment close to the real society.

A determination device according to one aspect of the present disclosure includes:
an acquisition unit configured to acquire a sightline direction of a user and a sightline direction of at least one facing person facing the user;
a first determination unit configured to determine whether a sightline of the user is directed to the facing person based on the sightline direction of the user acquired by the acquisition unit;
a second determination unit configured to deter mine whether a sightline of the facing person is directed to the user based on the sightline direction of the facing person acquired by the acquisition unit; and
an output unit configured to output a feedback to the user and/or an executor based on a result of a determination of the first determination unit and a result of a determination of the second determination unit.

A determination method according to one aspect of the present disclosure includes:
acquiring a sightline direction of a user and a sightline direction of at least one facing person;
performing a first determination of determining whether a sightline of the user is directed to the facing person based on the acquired sightline direction of the user;
performing a second determination of determining whether a sightline of the facing person is directed to the user based on the acquired sightline direction of the facing person; and
outputting a feedback to the user and/or an executor based on a result of the first determination and a result of the second determination.

A training method according to an aspect of the present disclosure includes using the determination method according to the aspect described above.

A program according to one aspect of the present disclosure is configured to cause a computer to execute the determination method according to the aspect described above.

### Advantageous Effects of Invention

With the determination device according to the aspect described above, it is possible to reproduce a sightline communication environment close to the real society. It is also possible to grasp a condition of a sightline communication of a user to perform a feedback to a user and/or an executor, or to quantify the sightline communication.

With the determination method and the program according to the aspect described above, it is possible to reproduce a sightline communication environment close to the real society. It is also possible to grasp a condition of a sightline communication of a user to perform a feedback to a user and/or an executor, or to quantify the sightline communication.

With the training method according to the aspect described above, a sightline communication environment close to the real society is reproduced using the determination method according to the aspect described above, and it becomes possible to conduct a highly effective training. Furthermore, it is possible to implement a training even when an executor (e.g., a doctor, a psychiatrist (a psychologist), a counselor, a therapist, or a supervisor of the training) is at a remote location, or while the executor is absent.

### BRIEF DESCRIPTION OF DRAWINGS

FIG. 1 is a block diagram illustrating a determination system including a determination device according to an embodiment of the present disclosure;

FIG. 2 is a first flowchart for explaining an example of a method for determining “whether the sightline of a user is directed to the facing person” and “whether the facing person's sightline is directed to the user”;

FIG. 3 is a diagram for explaining a first example of a method for determining “whether the user's sightline is directed to the facing person”;

FIG. 4 is a second flowchart for explaining an example of the method for determining “whether the sightline of a user is directed to the facing person” and “whether the facing person's sightline is directed to the user”;

FIG. 5 is a third flowchart for explaining an example of the method for determining “whether the sightline of a user is directed to the facing person” and “whether the facing person's sightline is directed to the user”;

FIG. 6 is a fourth flowchart for explaining an example of the method for determining “whether the sightline of a user is directed to the facing person” and “whether the facing person's sightline is directed to the user”;

FIG. 7 is a first diagram for explaining a fourth example in which it is determined “whether the user's sightline is directed to the facing person”;

FIG. 8 is a second diagram for explaining the fourth example in which it is determined “whether the user's sightline is directed to the facing person”;

FIG. 9 is a diagram illustrating an example of a feedback output from the determination device in FIG. 1;

FIG. 10 is a first flowchart for explaining an example of a feedback process performed by the determination device in FIG. 1;

FIG. 11 is a second flowchart for explaining the example of the feedback process performed by the determination device in FIG. 1;

FIG. 12 is a third flowchart for explaining the example of the feedback process performed by the determination device in FIG. 1; and

FIG. 13 is a fourth flowchart for explaining the example of the feedback process performed by the determination device in FIG. 1.

### DETAILED DESCRIPTION

An example according to the present disclosure will now be explained with reference to the accompanying drawings.

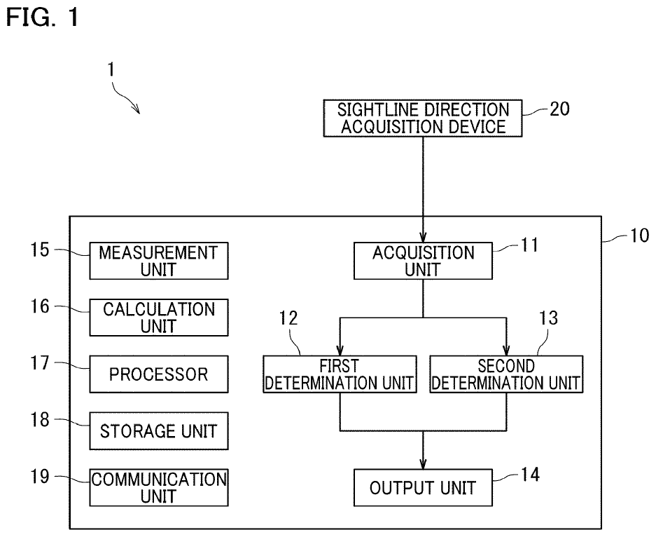

As illustrated in FIG. 1, a determination system 1 according to one embodiment of the present disclosure includes a determination device 10 and a sightline direction acquisition device 20. The sightline direction acquisition device 20 is configured to detect a sightline direction of a user, and a sightline direction of at least one facing person facing the user, and to output the detected sightline directions of the user and the facing person to the determination device 10. The determination system 1 may be configured to include two sightline direction acquisition device 20 and to detect the user's sightline direction and the facing person's sightline direction by the different sightline direction acquisition device 20. The user of the present disclosure includes a person who receives a sightline communication training. The facing person of the present disclosure is not limited to a person the user is actually facing, and includes an animal which the user is actually facing, a person, an animal, a character, or the like displayed on a display such as a television monitor, a tablet, a head-mounted display, a smartphone, or the like.

As illustrated in FIG. 1, the determination device 10 includes an acquisition unit 11, a first determination unit 12, a second determination unit 13, and an output unit 14. In the present embodiment, the determination device 10 includes a measurement unit 15, a calculation unit 16, a processor 17, a storage unit 18, and a communication unit 19. Each of the acquisition unit 11, the first determination unit 12, the second determination unit 13, the output unit 14, the measurement unit 15, and the calculation unit 16 is implemented by the processor 17 executing a predetermined program stored in the storage unit 18, for example. Examples of the processor 17 include a CPU, an MPU, a GPU, a DSP, an FPGA, an ASIC, and the like. The storage unit 18 is constituted of an internal recording medium or an external recording medium, for example. An example of the internal recording medium includes a nonvolatile memory. Examples of the external recording medium include a hard disk (HDD), a solid-state drive (SSD), an optical disk device, and the like. The communication unit 19 is constituted of a communication line or a communication module for transmitting and receiving data to and from external device such as a server.

The acquisition unit 11 is configured to acquire the sightline direction of the user and the sightline direction of the at least one facing person facing the user, for example, via the communication unit 19. In the present embodiment, the acquisition unit 11 is configured to acquire the sightline directions of the user and the facing person output from the sightline direction acquisition device 20.

The “sightline direction” acquired by the acquisition unit 11 is not limited to information related to the sightline direction of the user and the facing person, and also includes information with which the sightline direction of the user and the facing person can be calculated. The “sightline direction” includes, for example, information related to a sightline-position of the user and the facing person, information related to an image coming into view of the user or the facing person (hereinafter, referred to as a view image), and information related to an image of the user or the facing person displayed on a display of an electronic device such as a PC (hereinafter, referred to as a displayed image). The “information with which the sightline direction of the user and the facing person can be calculated” includes a roll angle, a pitch angle, and a yaw angle.

The first determination unit 12 is configured to make a first determination which determines whether the sightline of the user is directed to the facing person, based on the sightline direction of the user acquired by the acquisition unit 11. The second determination unit 13 is configured to make a second determination which determines whether the sightline of the facing person is directed to the user, based on the sightline direction of the facing person acquired by the acquisition unit 11.

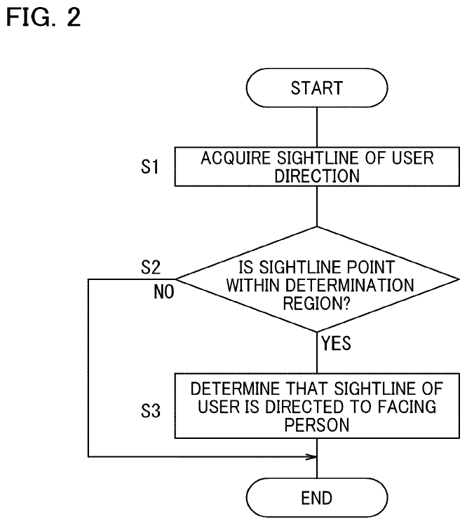

An example of a method for determining “whether the sightline of the user is directed to the facing person” and “whether the sightline of the facing person is directed to the user” will now be explained with reference to FIGS. 2 to 8. One of the following determination methods may be applied to both the first determination and the second determination, or any different combinations of these determination methods may be applied to the first determination and the second determination, respectively.

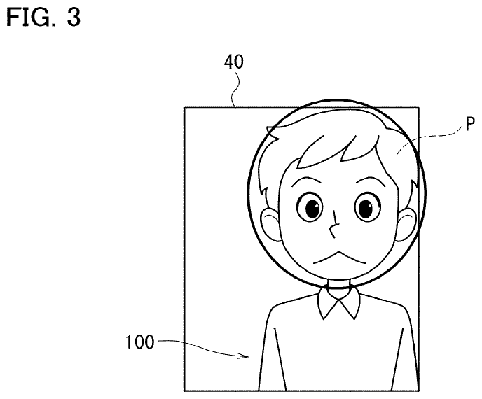

A first example of the method for determining “whether the sightline of the user is directed to the facing person” will now be explained with reference to FIGS. 2 and 3. In the first example, the sightline direction acquisition device 20 is constituted of a wearable terminal that is worn by the user or the facing person, that is capable of acquiring the sightline-position of the wearer, and that is capable of acquiring the view image of the wearer (e.g., a wearable eye tracking device), or a stationary device (e.g., a stationary eye tracking device). The process illustrated in FIG. 2 is performed by the processor 17 executing a predetermined program, for example.

As illustrated in FIG. 2, when the acquisition unit 11 acquires a sightline direction of a user acquired by the sightline direction acquisition device 20 (step S1), the first determination unit 12 determines whether an acquired sightline-position of the user is within a determination region P (see FIG. 3) (step S2).

The determination region P is set to a view image 40 of the user acquired by the sightline direction acquisition device 20, for example. For example, the face of the user or the facing person is recognized using a face detection algorithm, and the determination region P is automatically set thereto. In the process of recognizing a face of the user or the facing person, information of a position where the face of the user or the facing person is detected may be automatically normalized. When the determination region P is set to a part of the face such as eyes or a nose, the determination region P may be set using a landmark of the face. Alternatively, a part of the face may be set as a watch target, and the determination region P may be set to a predetermined area with respect to the watch target as a center, for example. The predetermined area is set based on, for example, a distance between centers of right and left eyeballs of the facing person, or a size of the face of the facing person that is based on the distance between the eyeballs.

When using information of the landmark of the face, information of a center of the landmark that is the watch target may be used. For example, when the watch target is set to an eye, the determination region P may be set to a circular shape centered on the landmark of the right eye and/or the left eye. At this time, as a factor for deciding an area of the circle, it is possible to refer to a distance between centers of the eyes based on landmark information of the eyes, or a distance of a face width near the eyes based on landmark information of a contour of the face.

When there arc a plurality of watch target settings (e.g., set to the left and the right eyes), the determination region P may be a union of the determination regions that are set correspondingly to the respective watch targets.

The determination region P may set for both of a main target and a sub-target. Specifically, when the watch target is “eyes”, the main target may be set based on landmark information of the “eyes”, and the sub-target may be set to a face frame based on landmark information of a contour of the face. It is possible to output different feedbacks (e.g., to give ranks to the feedbacks) depending on whether the sightline is within the main target or within the sub-target but outside the main target. By providing a feedback in the manner described above, an effect of training or therapy can be enhanced.

A lower limit value can be provided to a size of the determination region P. This is because it is difficult to capture a sightline-position of the user accurately due to influences of involuntary eye movement of the eyeballs and detection noise of a sightline sensor when the facing person in the view image 40 is too small, for reasons such as that the facing person is too far from the user, the facing person in a presented image is too small, the presented image is too small, or a screen presenting the presented image is too far from the user,

The lower limit value of the determination region P may be an arbitrary value determined in advance regardless of the user, or may be an arbitrary value that can be set at the time of use. The lower limit value of the determination region P may be a value determined based on sightline-position variation data when the user gazes at an arbitrary point. For example, it is possible to use a region of a circle having a radius which is twice the standard deviation (2SD) of sightline-position variation data, as the lower limit value of the determination region P. It is possible to expand a range of the region to three times the standard deviation (3SD) or four times the standard deviation (45D), depending on a training stage. The lower limit value of the determination region P may be determined for each user or may be determined from an average value over a plurality of users. However, it is preferable that the lower limit value of the determination region P is determined for each user in consideration of variations in involuntary eye movement of eyeballs among individuals. As a default value for the sightline-position determination device 10, an average value over a plurality of healthy persons can be set as the lower limit value of the determination region P. The sightline-position variation data means a plot of variation of the sightline position over time.

When the lower limit value is set to the determination region P, the determination region P is set so as not to fall below the lower limit value. For example, when a size of the determination region P is calculated based on a distance between centers of the right and left eyeballs and a size thereof is smaller than the lower limit value, the lower limit value is set as a size of the determination region P. In this manner, it is possible to determine a sightline match accurately without being affected by a size of the facing person or a distance to the facing person.

The determination region P (including the main target and/or the sub-target) may be set by a determination region setting unit (not illustrated). In this case, the determination device 10 includes the determination region setting unit, in addition to the acquisition unit 11, the first determination unit 12, the second determination unit 13, and the output unit 14. The determination region setting unit is configured to set the determination region P, and output information of the determination region P to the first determination unit 12 and/or the second determination unit 13.

When the sightline-position is determined to be within the determination region P, the first determination unit 12 determines that “the sightline of the user is directed to the facing person” (step S3), and the process is ended.

When it is not determined that the sightline-position is within the determination region P in step S2, the process is ended without determining that “the sightline of the user is directed to the facing person”.

By switching the “user” and the “facing person”, it is possible to use the process in FIG. 2 for determining “whether the sightline of the facing person is directed to the user”.

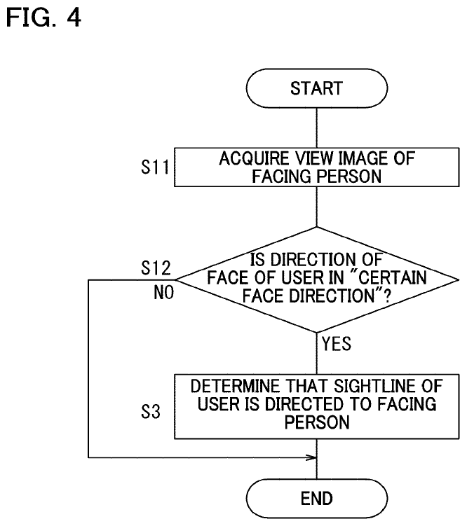

A second example of the method for determining “whether the sightline of the user is directed to the facing person” will be explained with reference to FIG. 4. In the second example, the sightline direction acquisition device 20 is constituted of a wearable terminal that is worn by the user or the facing person and that is capable of acquiring a view image of a wearer (e.g., a wearable camera), or a stationary device (e.g., a mount camera or a built-in camera on a PC). A process illustrated in FIG. 4 is performed by the processor 17 executing a predetermined program, for example.

As illustrated in FIG. 4, when the acquisition unit 11 acquires a view image of the facing person acquired by the sightline direction acquisition device 20 (step S11), the first determination unit 12 calculates a direction of the face of the user from the acquired view image of the facing person, and determines whether the calculated direction of the face of the user is in a “certain face direction” (step S12).

The “certain face direction” is a range of directions of the face where a face of the facing person can be looked without consciously moving the eyes. For example, using a direction in which a face of the user in a view image of the facing person directly faces the facing person as a reference direction, when an angle in a left and right direction formed by the calculated direction of the face of the user with respect to the reference direction is within a range of ±30 degrees and an angle in an up and down direction formed by the calculated direction of the face of the user with respect to the reference direction is within a range of ±5 degrees, the calculated direction of the face of the user is determined as being in the “certain face direction”.

When the calculated direction of the face of the user is determined to be in the “certain face direction”, the first determination unit 12 determines that “the sightline of the user is directed to the facing person” (step S3), and the process is ended.

When it is determined in step S12 that the direction of the face of the user is not in the “certain face direction”, the process is ended without determining that “the sightline of the user is directed to the facing person”.

By switching the “user” and the “facing person”, the process in FIG. 4 can be applied to a situation for determining whether the sightline of the “facing person” is directed to the user.

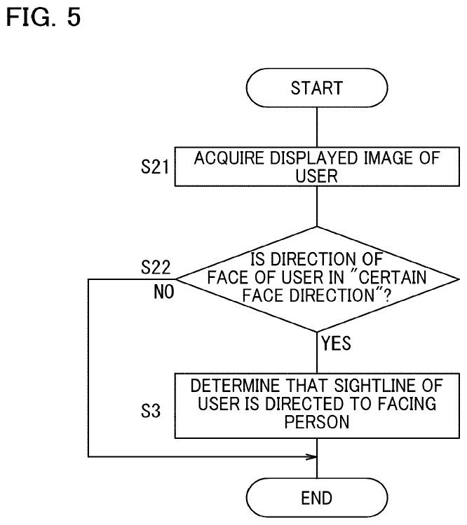

A third example in which “whether the sightline of the user is directed to the facing person” is determined will be explained with reference to FIG. 5. In the third example, the sightline direction acquisition device 20 includes a display on which a user or a facing person is displayed, and is constituted of an electronic device such as a PC capable of acquiring a displayed image of a person being displayed on the display. The sightline direction acquisition device 20 may be incorporated in the determination device 10. A process illustrated in FIG. 5 is performed by the processor 17 executing a predetermined program, for example.

As illustrated in FIG. 5, when the acquisition unit 11 acquires the displayed image of the user acquired by the sightline direction acquisition device 20 (step S21), the first determination unit 12 calculates a direction of the face of the user from the acquired displayed image of the user, and determines whether the calculated direction of the face of the user is in the “certain face direction” (step S22).

When the calculated direction of the face of the user is determined to be in the “certain face direction”, the first determination unit 12 determines that “the sightline of the user is directed to the facing person” (step S3), and the process is ended.

When it is determined in step S22 that the direction of the face of the user is not in the “certain face direction”, the process is ended without determining that “the sightline of the user is directed to the facing person”.

By switching the “user” and the “facing person”, the process in FIG. 5 can be applied to a situation for determining whether the sightline of the “facing person” is directed to the user.

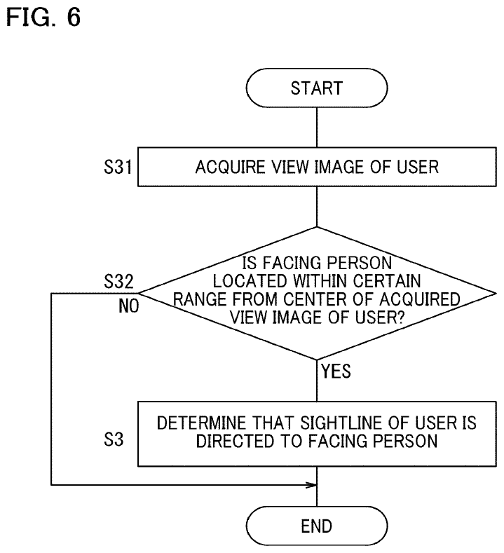

A fourth example in which “whether the sightline of the user is directed to the facing person” is determined will be explained with reference to FIGS. 6 to 8. In the fourth example, the sightline direction acquisition device 20 is constituted of a wearable terminal that is worn by the user or the facing person and that is capable of acquiring a view image of a wearer (e.g., a wearable camera), or a stationary device (e.g., a mount camera or a built-in camera on a PC). A process illustrated in FIG. 6 is performed by the processor 17 executing a predetermined program, for example.

As illustrated in FIG. 6, when the acquisition unit 11 acquires a view image of the user acquired by the sightline direction acquisition device 20 (step S31), the first determination unit 12 determines whether the facing person is located within a certain range from a center of the acquired view image of the user (step S32). For example, when a certain percentage of the face of the facing person is within a certain range or more, it is determined that “the facing person is within the certain range”. The “certain range” and the “certain percentage” may be set in any way. For example, 80% of the “certain range” may be set as the “certain percentage”.

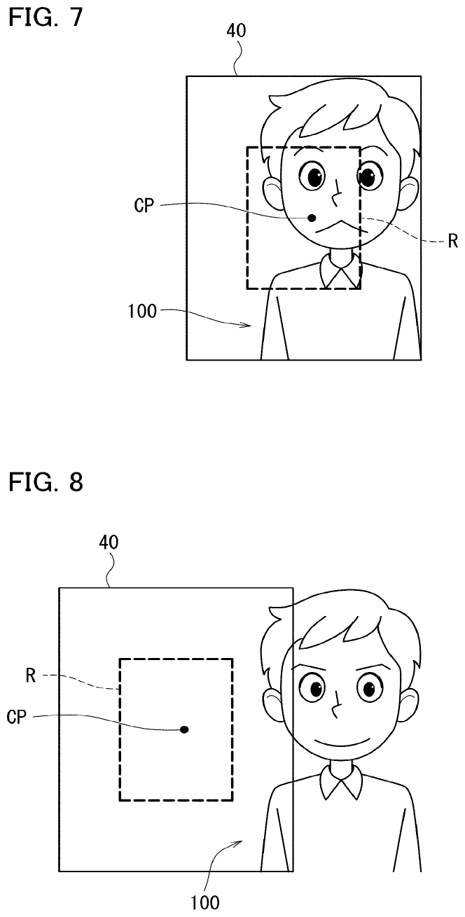

FIGS. 7 and 8 illustrate examples of the acquired view image of the user. In FIGS. 7 and 8, a certain range R indicated by a broken line is positioned around a central point CP of a view image 40 of the user. In FIG. 7, a facing person 100 positions inside the certain range R, but in FIG. 8, the facing person 100 positions outside of the certain range R.

When the facing person is determined to be within the certain range from a center of the acquired view image of the user (see FIG. 7), the first determination unit 12 determines that “the sightline of the user is directed to the facing person” (step S3), and the process is ended.

When it is not determined that the facing person is within the certain range from the center of the view image of the user acquired in step S32 (see FIG. 8), the process is ended without determining that “the sightline of the user is directed to the facing person”.

By switching the “user” and the “facing person”, the process in FIG. 6 can be applied to a situation for determining whether the sightline of the “facing person” is directed to the user.

When there are a plurality of facing persons, for example, “whether the user's sightline is directed to the facing person” and “whether the facing person's sightline is directed to the user” are determined for some or all of the plurality of facing persons. For example, assuming that the user is facing a first facing person and a second facing person, as the facing persons. In this situation, the first determination unit 12 may either determine whether the sightline of the user is directed to the first facing person, or determine whether the sightline of the user is directed to the first facing person and whether the sightline of the user is directed to the second facing person. The second determination unit 13 may either determine whether the sightline of the first facing person is directed to the user, or determine whether the sightline of the first facing person is directed to the user and whether the sightline of the second facing person is directed to the user.

For identifications of the plurality of facing persons, for example, an identification method with ID fixation using identification markers such as ArUco markers may be used. With this identification method, for example, each of the facing persons wears an identification marker on the right chest. When the identification marker closest to a right eye of a facing person satisfies the following determination condition, the ID is then fixed as the identification marker of the facing person.

Determination condition: a straight line connecting the identification marker and (a center point of) the right eye of the facing person forms an angle within a certain range (e.g., 50 degrees to 100 degrees) with a horizontal line passing through the identification marker and extending toward the left chest.

The output unit 14 is configured to output a feedback to the user and/or the executor, based on a result of a determination of the first determination unit 12 and a result of a determination of the second determination unit 13.

For example, assuming that the user is facing the first facing person and the second facing person as the facing persons, and each of the first determination unit 12 and the second determination unit 13 determines for both of the first facing person and the second facing person. In this case, the output unit 14 outputs a feedback to the user based on the determination results of the first determination unit 12 and the second determination unit 13 for the first facing person, and outputs a feedback to the user based on the determination results of the first determination unit 12 and the second determination unit 13 for the second facing person.

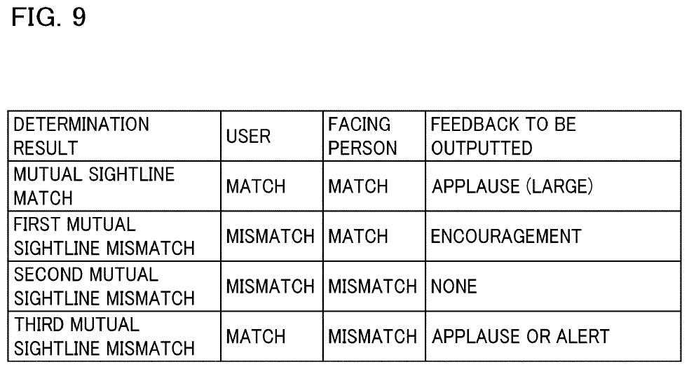

For example, as illustrated in FIG. 9, the feedback to be output is decided based on whether the sightline of the user is directed to the facing person and whether the sightline of the facing person is directed to the user. In FIG. 9, “match” means a condition in which a predetermined maintaining time has elapsed while keeping determining that the sightline of the user is directed to the facing person, or in which a predetermined maintaining time has elapsed while keeping determining that the sightline of the facing person is directed to the user. “Mismatch” means a condition in which there has been no determination that the sightline of the user is directed to the facing person, or a condition in which the predetermined maintaining time has not elapsed while keeping determining that the sightline of the user is directed to the facing person, or a condition in which there has been no determination that the sightline of the facing person is directed to the user, or a condition in which the predetermined maintaining time has not elapsed while keeping determining that the sightline of the facing person is directed to the user. The maintaining time may be arbitrarily set. For example, the maintaining time may be a predetermined time (e.g., 3.0 seconds) or may be a predetermined range of time (e.g., 2.5 seconds to 4.0 seconds, or 3.0 seconds to 3.5 seconds).

For example, as illustrated in FIG. 9, when both of the user and the facing person are determined to “match” (hereinafter, referred to as a “mutual sightline match”), an applause (large) is output as a feedback. When the user is determined to “mismatch” but the facing person is determined to “match” (hereinafter, referred to as a “first mutual sightline mismatch”), an encouragement is output as a feedback. When both of the user and the facing person is determined to “mismatch” (hereinafter, referred to as a “second mutual sightline mismatch”), no feedback is output. When the user is determined to “match” but the facing person is determined to “mismatch” (hereinafter, referred to as a “third mutual sightline mismatch”), an applause (small) or an alert is output as a feedback. The “third mutual sightline mismatch” includes a condition in which the user has kept seeing at eyes of the facing person for an excessively long time (hereinafter, referred to as sightline perseveration). The alert is output as feedback, for example, when the condition of the “third mutual sightline mismatch” has been maintained for time making the facing person feel uncomfortable or longer. Specifically, when the condition of the “third mutual sightline mismatch” has been maintained for a predetermined time length (e.g., 6 seconds), an alert is output.

The feedback is performed to the user and/or the executor through a sensation selected from a visual sensation, an auditory sensation, and a tactile sensation. Each of the applause (large), the applause (small), the alert, and the encouragement, for example, is selected from one of an image including letters, symbols, and a picture; a sound including voice; a vibration; and a combination thereof, and is output in real-time via a feedback output device, e.g., a display and/or a speaker. The feedback output device may be a part of the determination device 10 or may not be a part of the determination device 10.

The feedback may be decided depending on whether the user has been able to perform an instruction of a presented task. For example, when the user has been able to perform the instruction, an applause is output as the feedback, and, when the user has failed to perform the instruction, an encouragement is output as the feedback.

For example, for an instruction “when facing person is looking at your eyes, make an eye contact with the facing person”, when getting “mutual sightline match”, an applause is output as the feedback, and when getting “first mutual sightline mismatch”, an encouragement is output as the feedback. For an instruction “when your facing person is not looking at your eyes, do not make an eye contact with the facing person”, when getting “second mutual sightline mismatch”, an applause is output as the feedback, and when getting “third mutual sightline mismatch”, an encouragement and/or an alert is/are output as the feedback. For an instruction “even if your facing person is not looking at your eyes, make an eye contact with the facing person”, when getting “third mutual sightline mismatch”, an applause is output as the feedback, and when getting “second mutual sightline mismatch”, an encouragement is output as the feedback.

The output unit 14 may output the following types of feedbacks.
A degree of avoidance that the user avoids a mutual sightline match with the facing person
A mutual sightline match/mismatch pattern of the user with the facing person
A degree of sightline perseveration
Match time, which will be described later
First mismatch time, which will be described later
Second mismatch time, which will be described later
Third mismatch time, which will be described later

The “degree of avoidance” to be feedback is not limited to an applause or the like output in real-time during the use of the determination device 10, but also includes an analysis based on an accumulated result of a repeated determination method (e.g., a series of trainings) with the determination device 10. In other words, the “feedback” includes “feedback performed every use (real-time feedback)” and “feedback analyzed the results so far together (feedback of the accumulated results)”. The accumulated results from the determination method may be stored in the storage unit 18 or may be stored in an external device, for example. The analysis based on the accumulated results may be performed by the calculation unit 16, or by an external device, for example. Transmission and reception of data between the determination device 10 and the external device are performed via the communication unit 19.

The measurement unit 15 measures the following times, for example.
A time that a mutual sightline match is maintained (hereinafter, referred to as a match time), where the mutual sightline match is a condition the sightline of the user is determined as being directed to the facing person and the sightline of the facing person is determined as being directed to the user.
A time that a first mutual sightline mismatch is maintained (hereinafter, referred to as a first mismatch time), where the first mutual sightline mismatch is a condition in which the sightline of the user is not determined as being directed to the facing person, but the sightline of the facing person is determined as being directed to the user.
A time that a second mutual sightline mismatch is maintained (hereinafter, referred to as a second mismatch time), where the second mutual sightline mismatch is a condition in which neither the sightline of the user is determined as being directed to the facing person nor the sightline of the facing person is determined as being directed to the user.
A time that a third mutual sightline mismatch is maintained (hereinafter, referred to as a third mismatch time), where the third mutual sightline mismatch is a condition in which the sightline of the user is determined as being directed to the facing person, but the sightline of the facing person is not determined as being directed to the user.

In the present embodiment, the measurement unit 15 serves as a first measurement unit, a second measurement unit, and a third measurement unit. In other words, the measurement unit 15 is an example of the first measurement unit, an example of the second measurement unit, and an example of the third measurement unit.

The calculation unit 16 calculates the following degrees, for examples.
A degree of avoidance that the user avoids the mutual sightline match with the facing person, and that is based on the first mismatch time.
A degree of avoidance that the user avoids the mutual sightline match with the facing person, and that is based on the second mismatch time.
A degree of sightline perseveration based on that third mismatch time.

The degree of avoidance means that when the degree of avoidance is higher, the user is more likely to avoid the mutual sightline match. The degree of avoidance of the mutual sightline match becomes higher when an accumulated time or a maximum duration of the first mismatch time becomes longer, for example.

In the present embodiment, the calculation unit 16 serves as a first calculation unit, a second calculation unit, and a third calculation unit. In other words, the calculation unit 16 is an example of the first calculation unit, an example of the second calculation unit, and an example of the third calculation unit.

For example, the calculation unit 16 may calculate the mutual sightline match/mismatch pattern based on various combinations of the match time, the first mismatch time, the second mismatch time, and the third mismatch time.

The mutual sightline match/mismatch pattern is a result of calculating a pattern that is dominant in the user, and may be classified into “a pattern in which the match time is short, and the first mismatch time, the second mismatch time, and the third mismatch time are long” and a “pattern in which the match time and the third mismatch time are long”, for example. The mutual sightline match/mismatch pattern may be calculated using the accumulated time of the match time and the respective types of the mismatch time and the maximum duration of the match time and the respective types of the mismatch time, for example.

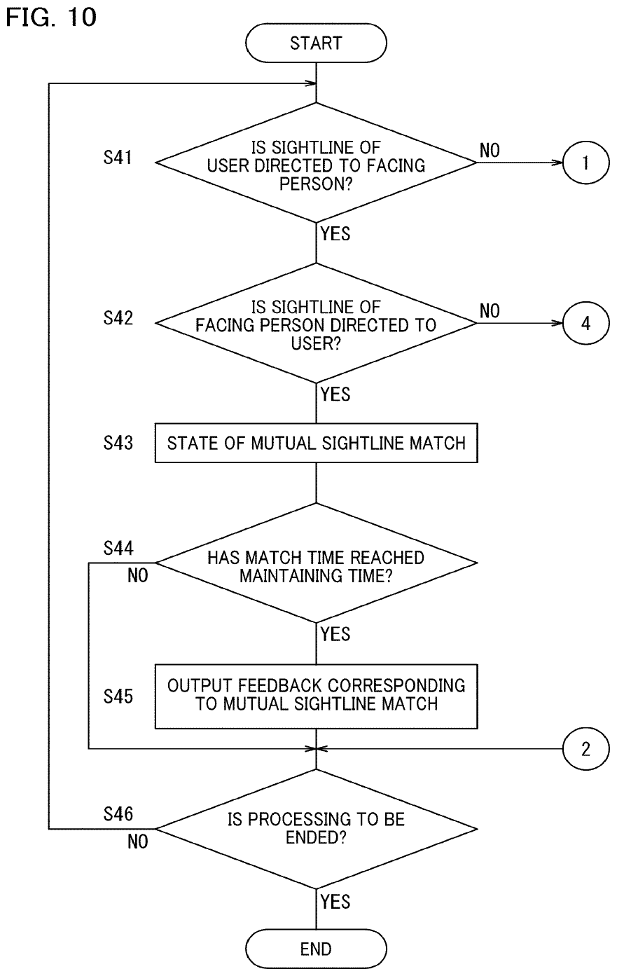

An example of a feedback deciding process will now be explained with reference to FIGS. 10 to 13. The process illustrated in FIGS. 10 to 13 is performed by the processor 17 executing a predetermined program, for example.

As illustrated in FIG. 10, the first determination unit 12 determines whether the sightline of the user is directed to the facing person based on the sightline direction of the user acquired by the acquisition unit 11 (step S41). When the sightline of the user is determined as being directed to the facing person, the second determination unit 13 determines whether the sightline of the facing person is directed to the user based on the sightline direction of the facing person acquired by the acquisition unit 11 (step S42).

When it is determined in step S42 that the sightline of the facing person is directed to the user, the determination device 10 determines that it is a state of the mutual sightline match (step S43), and determines whether the match time has reached a preset maintaining time (step S44). When it is determined that the match time has reached the maintaining time, the output unit 14 outputs a feedback corresponding to the “mutual sightline match” (step S45).

When it is not determined in step S44 that the match time has reached the maintaining time, or when the feedback has been output in step S45, the output unit 14 determines whether the feedback deciding process is to be ended (step S46). When it is determined that the feedback deciding process is to be ended, the feedback deciding process is ended. When it is not determined that the feedback deciding process is to be ended, the process returns to step S41, and whether the sightline of the user is directed to the facing person is determined.

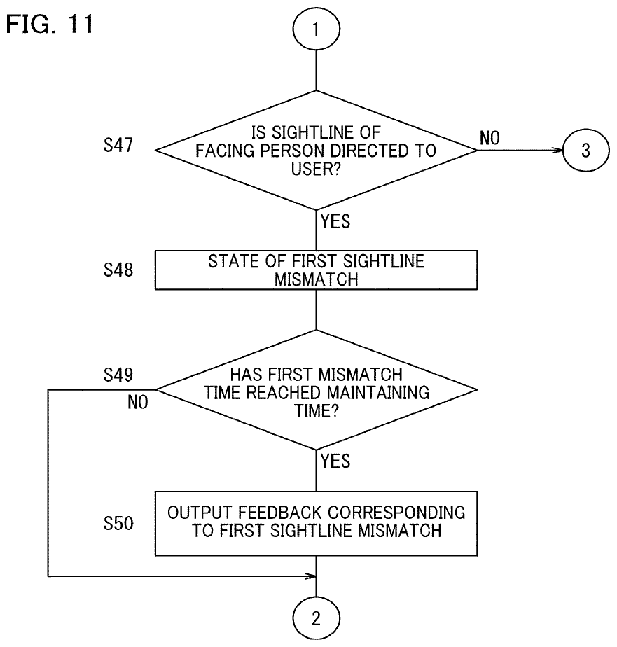

When it is not determined in step S41 in FIG. 10 that the sightline of the user is directed to the facing person, as illustrated in FIG. 11, the second determination unit 13 determines whether the sightline of the facing person is directed to the user based on the sightline direction of the facing person acquired by the acquisition unit 11 (step S47).

When it is determined in step S47 that the sightline of the facing person is directed to the user, the determination device 10 determines that it is a state of the first sightline mismatch (step S48), and determines whether the first mismatch time has reached a preset maintaining time (step S49). When the first mismatch time is determined as having reached the maintaining time, the output unit 14 outputs a feedback corresponding to the “first mutual sightline mismatch” (step S50).

When it is not determined in step S49 that the first mismatch time has reached the maintaining time, or when a feedback has been output in step S50, the process is shifted to step S46.

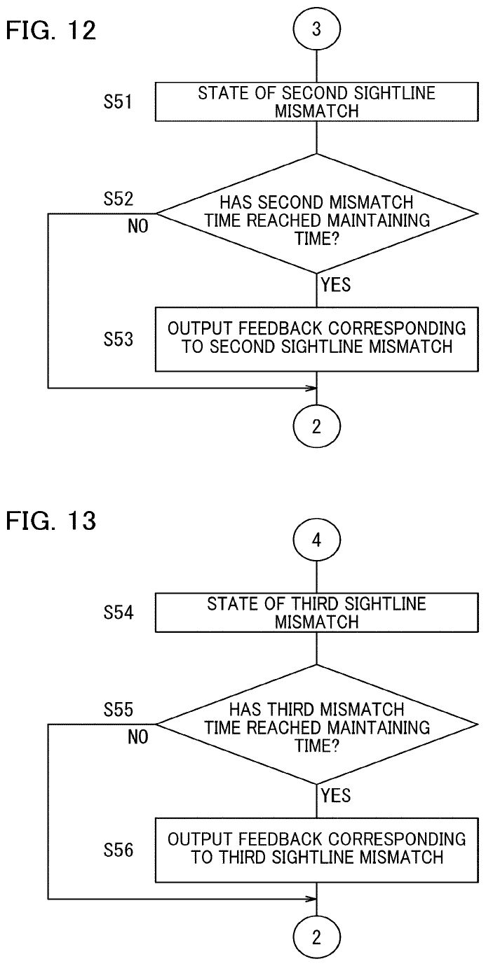

When it is not determined in step S47 in FIG. 11 that the sightline of the facing person is directed to the user, as illustrated in FIG. 12, the determination device 10 determines that it is a state of the second sightline mismatch (step S51), and determines whether the second mismatch time has reached a preset maintaining time (step S52). When it is determined that the second mismatch time has reached the maintaining time, the output unit 14 outputs a feedback corresponding to the “second mutual sightline mismatch” (step S53).

When it is not determined in step S52 that the second mismatch time has reached the maintaining time, and when a feedback has been output in step S53, the process is shifted to step S46.

When it is not determined in step S42 in FIG. 10 that the sightline of the facing person is directed to the user, the determination device 10 determines that it is a state of the third sightline mismatch (step S54), as illustrated in FIG. 13, and determines whether the third mismatch time has reached a preset maintaining time (step S55). When it is determined that the third mismatch time has reached the maintaining time, the output unit 14 outputs a feedback corresponding to the “third mutual sightline mismatch” (step S56).

When it is not determined in step S55 that the third mismatch time has reached the maintaining time, or when a feedback has been output in step S56, the process is shifted to step S46.

The maintaining time in step 44, the maintaining time in step S49, the maintaining time in step S52, and the maintaining time in step S55 may be set to arbitrary time. For example, when time in use or the number of times in use is short or small, the maintaining time may be set short. The maintaining time may be extended as time in use or the number of times in use becomes increased. By keeping the maintaining time short, the feedbacks corresponding to the “mutual sightline match”, the “first mutual sightline mismatch”, the “second mutual sightline mismatch”, and the “third mutual sightline mismatch” are output more easily. By keeping the maintaining time long, the feedbacks corresponding to the “mutual sightline match”, the “first mutual sightline mismatch”, the “second mutual sightline mismatch”, and the “third mutual sightline mismatch” are output less easily.

In addition to the series of steps described above, or instead of some of the steps described above, a step of determining whether the user has been able to do an instruction for the presented task may be performed and the feedback may be determined depending on a result of the determination.

The determinations in step S43, step S48, step S51, and step S54 may be made by an arbitrary configuration of the deter urination device 10. The arbitrary configuration may be any one of the first determination unit 12, the second determination unit 13, and the output unit 14, or may be another determination unit separate from the first determination unit 12 and the second determination unit 13, for example.

With the determination device 10 and the determination system 1, the following advantageous effects can be achieved.

The determination device 10 includes the acquisition unit 11, the first determination unit 12, the second determination unit 13, and the output unit 14. The acquisition unit 11 is configured to acquire the sightline direction of the user and the sightline direction of at least one facing person facing the user. The first determination unit 12 is configured to determine whether the sightline of the user is directed to the facing person based on the sightline direction of the user acquired by the acquisition unit 11. The second determination unit 13 is configured to determine whether the sightline of the facing person is directed to the user based on the sightline direction of the facing person acquired by the acquisition unit 11. The output unit 14 is configured to output a feedback to the user and/or the executor based on the result of the determination of the first determination unit 12 and the result of the determination of the second determination unit 13. With such a configuration, it is possible to reproduce a sightline communication environment close to the real society. In addition, the user and/or the executor can grasp a condition of a sightline communication of the user objectively and quantitatively. The determination device 10 outputs a feedback based on the result of the determination of the first determination unit 12 and the result of the determination of the second determination unit 13. Therefore, when using the determination device 10 in a therapy or a training of a neuropsychiatric disorder, for example, it is possible to enhance motivation of the user for the therapy or the training.

The determination system 1 includes the determination device 10 and the sightline direction acquisition device 20 that is configured to detect the sightline directions of the user and the facing person, and to output the detected sightline directions of the user and the facing person to the determination device 10. It is possible to implement the determination system 1 capable of reproducing a sightline communication environment close to the real society by the determination device 10.

The determination device 10 may use any one or more configurations among a plurality of configurations described below. In other words, any one or more configurations among the following plurality of configurations may be removed when the configurations are included in the embodiment, and may be added when the configurations are not included in the embodiment. By using such a configuration, it is possible to reproduce a sightline communication environment closer to the real society, more reliably.

The determination device 10 includes the measurement unit 15 (an example of a first measurement unit) configured to measure the first mismatch time.

The determination device 10 includes the calculation unit 16 (an example of a first calculation unit) configured to calculate a degree of avoidance that the user avoids the mutual sightline match with the facing person based on the first mismatch time measured by the measurement unit 15. The output unit 14 outputs the degree of avoidance calculated by the calculation unit 16 as a feedback. In this manner, it is possible to quantify the degree of avoidance of sightline match with the other (e.g., a degree of scopophobia with which a person exhibits a tendency to make behaviors such as facing and looking downwards in front of people). The calculation unit 16 can calculate a mutual sightline match/mismatch pattern based on the first mismatch time measured by the measurement unit 15. The output unit 14 can output the mutual sightline match/mismatch pattern calculated by the calculation unit 16 as a feedback. In this manner, the mutual sightline match/mismatch pattern (e.g., the pattern of behaviors the user wants to avoid) can be quantified. With quantifying the degree of avoidance of sightline match with the other, and quantifying the mutual sightline match/mismatch pattern that is dominant in the user, the determination device 10 may be used as a diagnosis aiding tool, for example. By selecting an instruction or a task based on the calculated results, an effective training can be done.

The output unit 14 outputs the first mismatch time measured by the measurement unit 15 as a feedback.

The determination device 10 includes the measurement unit 15 (an example of a second measurement unit) configured to measure the second mismatch time. With such a configuration, it is possible to make adjustments between recognition of the user concerning the sightline of the other person and reality. For example, the recognition of the user related to the sightline of the other person is stored during or after training without giving a feedback in real-time during training, and is compared with the first sightline mismatch time, so that the recognition and a deviation with respect to reality is visualized for an executor (e.g., a medical personnel). Thus, the determination device 10 can use as a diagnosis aiding tool.

The determination device 10 includes the calculation unit 16 (an example of a second calculation unit) configured to calculate a degree of avoidance that the user avoids the mutual sightline match with the facing person based on the second mismatch time measured by the measurement unit 15. The output unit 14 outputs this degree of avoidance calculated by the calculation unit 16 as a feedback.

The output unit 14 outputs the second mismatch time measured by the measurement unit 15 as a feedback.

The determination device 10 includes the measurement unit 15 (an example of a third measurement unit) configured to measure the third mismatch time. With such a configuration, the degree of sightline perseveration can be quantified.

The determination device 10 includes the calculation unit 16 (an example of a third calculation unit) configured to calculate a degree of sightline perseveration that is a condition in which the user keeps seeing at the facing person excessively, based on the third mismatch time measured by the measurement unit 15. The output unit 14 outputs the degree of sightline perseveration calculated by the calculation unit 16 as a feedback.

The output unit 14 outputs the third mismatch time measured by the measurement unit 15 as a feedback.

The output unit 14 outputs a different feedback for each of the mutual sightline match, the first mutual sightline mismatch, the second mutual sightline mismatch, and the third mutual sightline mismatch. In other words, because the feedback is output depending on whether the user and the facing person are looking at each other, highly effective sightline communication training can be implemented based on an appropriate feedback.

Assuming that the user is facing at least a first facing person and a second facing person, as the facing person. The first determination unit 12 is configured to determine whether the sightline of the user is directed to the first facing person, and determine whether the sightline of the user is directed to the second facing person. The second determination unit 13 is configured to determine whether the sightline of the first facing person is directed to the user, and determine whether the sightline of the second facing person is directed to the user. The output unit 14 is configured to output a feedback to the user based on the determination results of the first determination unit 12 and the second determination unit 13 for the first facing person, and output a feedback to the user based on the determination results of the first determination unit 12 and the second determination unit 13 for the second facing person. With such a configuration, it is possible to quantify the number of times and the time in which the “mutual sightline match” is achieved for each of facing persons, and it is possible to implement a training for looking at a plurality of facing persons, for example. This training may be used, for example, as a therapy for what is called “look-aside fear” that is a fear in which a user is able to direct his/her eyes to a speaking person but is not able to or unsure whether it is alright to direct his/her eyes to another person who is near the speaking person. An example of abilities that can be quantified by the above configuration will be described below. Because a plurality of abilities can be quantified by the above configuration, it is possible to implement various types of training suitable for the situations individual users feel the urge to avoid. In addition, by quantifying a plurality of abilities, a user or an executor such as a medical executor can check the plurality of abilities according to numerics.
An ability to look at a person other than a speaking person in a setting of conversation with a plurality of facing persons. This ability serves as an index of the degree of so-called “look-aside fear”, which is a type of social anxiety, and it can be determined that the degree of the look-aside fear is high when this ability is low.
An ability to switch the sightline from a person looking at the user to a person not looking at the user in a setting of conversation with a plurality of facing persons.
An ability to find a person who is looking at the user and to match the sightline in the situation in which there are a plurality of facing persons.
An ability to match the sightline equally across all of a plurality of facing persons who are looking at the user in a situation where the user speaks in front of the plurality of facing persons.

The types of sightline phobias can be classified by a correlation analysis on under which a degree of anxiety (for example, monitoring by a heart rate or changes in the heart rate) increases in particular when it is one of the states of the mutual sightline match, the first sightline mismatch, and the third sightline mismatch. Therefore, it is possible to use the determination device 10 as a diagnosis aiding tool.

With the determination method, the treating method, the training method, the evaluation method, and the monitoring method according the present disclosure, the following advantageous effects can be achieved.

The determination method according to the present disclosure includes the steps described below. With such a configuration, it is possible to reproduce a sightline communication environment closer to real society. Furthermore, in the determination method according to the present disclosure, a feedback is output based on the results of the first determination and the result of the second determination. Therefore, when using the determination method according to the present disclosure in a therapy or a training of a neuropsychiatric disorder, for example, it is possible to enhance motivation of the user for the therapy or the training. In addition, the user and/or the executor can grasp a condition of a sightline communication of the user objectively and quantitatively.
Acquiring the sightline direction of the user and the sightline direction of at least one facing person.
Performing a first determination for determining whether the sightline of the user is directed to the facing person based on the acquired sightline direction of the user.
Performing a second determination for determining whether the sightline of the facing person is directed to the user based on the acquired sightline direction of the facing person.
Outputting a feedback to the user and/or the executor based on the result of the first determination and the result of the second determination.

The determination method according to the present disclosure may also be applied to a method for diagnosing neuropsychiatric disorders, and a method for aiding a diagnosis of neuropsychiatric disorders. In the present disclosure, the neuropsychiatric disorders include psychiatric disorders and neurodegenerative disorders.

Psychiatric disorders to which the present disclosure may be applied are not particularly limited, and include, for example, neurodevelopmental disorders, schizophrenia spectrum disorder and other psychotic disorders, anxiety disorders, obsessive-compulsive and related disorders, trauma- and stressor-related disorders, and neurocognitive disorders, according to the diagnostic criteria of DSM-5. Such psychiatric disorders include disorders and the like which tend to deteriorate the sightline communication ability along with psychiatric disorders. Such psychiatric disorders are not particularly limited, but preferably include neurodevelopmental disorders, anxiety disorders, obsessive-compulsive and related disorders, and trauma- and stressor-related disorders, and more preferably include neurodevelopmental disorders and anxiety disorders.

Disorders included in neurodevelopmental disorders are not particularly limited, and include, for example, intellectual disability, global developmental delay, language disorder, speech sound disorder, childhood-onset fluency disorder, social (pragmatic) communication disorder, unspecified communication disorders, autism spectrum disorder, attention-deficit hyperactivity disorder, specific learning disorder, developmental coordination disorder, stereotypic movement disorder, Tourette's disorder, persistent (chronic) motor or vocal tic disorder, provisional tic disorder, other specified tic disorder, unspecified tic disorder, other specified neurodevelopmental disorder, and unspecified neurodevelopmental disorder. Such disorders included in neurodevelopmental disorders are preferably language disorder, speech sound disorder, childhood-onset fluency disorder, social (pragmatic) communication disorder, unspecified communication disorder, autism spectrum disorder, attention-deficit hyperactivity disorder, specific learning disorder, stereotypic movement disorder, Tourette's disorder, persistent (chronic) motor or vocal tic disorder, provisional tic disorder, other specified tic disorder, unspecified tic disorder, other specified neurodevelopmental disorder, and unspecified neurodevelopmental disorder, and are more preferably childhood-onset fluency disorder and autism spectrum disorder.

Disorders included in schizophrenia spectrum and other psychotic disorders are not particularly limited, and include, for example, schizotypal personality disorder, delusional disorder, brief psychotic disorder, schizophreniform disorder, schizophrenia, schizoaffective disorder, substance/medication-induced psychotic disorder, psychotic disorder due to another medical condition, catatonia associated with another mental condition , catatonic disorder due to another medical condition, unspecified catatonia, other specified schizophrenia spectrum disorders and other psychotic disorders, unspecified schizophrenia spectrum disorders and other psychotic disorders, and attenuated psychosis syndrome (quasi-psychotic syndrome). Such disorders included in schizophrenia spectrum disorder and other psychotic disorders are preferably schizotypal personality disorder, schizophrenia, and schizoaffective disorder.

Disorders included in anxiety disorders are not particularly limited, and include, for example, separation anxiety disorder, selective mutism, specific phobia, social anxiety disorder, panic disorder, agoraphobia, generalized anxiety disorder, substance/medication-induced anxiety, anxiety disorder due to another medical condition, and other specified anxiety disorder. Such disorders included in anxiety disorders are preferably separation anxiety disorder, selective mutism, specific phobia, social anxiety disorder, panic disorder, agoraphobia, generalized anxiety disorder, and are more preferably social anxiety disorder.

Disorders included in obsessive-compulsive and related disorders, and trauma-and stressor-related disorders are not particularly limited, and include, for example, obsessive-compulsive disorder, body dysmorphic disorder, hoarding disorder, trichotillomania, excoriation disorder, substance/medication-induced obsessive-compulsive and related disorders, obsessive-compulsive and related disorders due to another medical condition, other specified obsessive-compulsive and related disorders, unspecified obsessive-compulsive and related disorder. Such disorders included in obsessive-compulsive and related disorders and trauma- and stressor-related disorders are preferably obsessive-compulsive disorder, body dysmorphic disorder, trichotillomania, excoriation disorder, and are more preferably body dysmorphic disorder.

Disorders included in trauma- and stressor-related disorders are not particularly limited, and include, for example, reactive attachment disorder, disinhibited social engagement disorder, posttraumatic stress disorder, acute stress disorder, adjustment disorders, other specified trauma- and stressor-related disorders, and unspecified trauma-and stressor-related disorder.

Disorders included in neurocognitive disorders are not particularly limited, and include, for example, delirium, dementia, and mild neurocognitive disorder. Such disorders included in neurocognitive disorders are preferably dementia, and mild neurocognitive disorder.

Neurodegenerative disorders are not particularly limited, and include, for example, Parkinson's disease, cranial neuropathy, and/or other disorders which tend to deteriorate the sightline communication ability.

The sightline communication ability is an index of an ability to communicate through a sightline in an interpersonal relationship in the real society. The sightline communication ability is constituted by a social sightline control ability (an ability to control and use a sightline as a signals), a social sightline understanding ability (an ability to understand a sightline as a signal), a social sightline transmission ability (an ability to transmit a sightline as a signal in an appropriate context), a social sightline response ability (an ability to respond to a sightline as a signal in an appropriate context), and the like. Examples of the social sightline control ability include a social sightline coincidence ability, a social sightline shifting ability, and a conjugate gaze ability. These can be measured through training according to the present disclosure (see Table 1). It is possible to comprehensively evaluate the sightline communication ability at time of consultation while observing a relevance to clinical scores of the Liebowitz Social Anxiety Scale, the Social Phobia Inventory, the Patient Health Questionnaire-9, and the Generalized Anxiety Disorder-7. However, the sightline communication ability may also be evaluated based on any one of or a combination of two or more of indices for determining the social sightline control ability (such as a determination of the mutual sightline match, a sightline distance between the user and the facing person, the number of times or an amount of time of mutual sightline match, the number of times or an amount of time of the first mutual sightline mismatch, the number of times or an amount of time of the second mutual sightline mismatch, and an amount of time of the third mutual sightline mismatch, where the amount of time includes an amount of time per determination, an average, and an amount of accumulated time). For example, the sightline communication ability may be comprehensively evaluated at time of consultation by using a combination of a clinical score and an index for determining the social sightline control ability.

The determination device, the determination method, the treating method, the training method, the evaluation method, or the monitoring method according to the present disclosure can also evaluate the social sightline understanding ability (the ability to understand a sightline as a signal) and the social sightline response ability (the ability to respond to a sightline as a signal in an appropriate context). The social sightline understanding ability can be evaluated based on whether the user understands whether the facing person is looking at the user. The social sightline response ability can be evaluated based on whether the user can take a pattern of actions such as to match the sightline of the user with the facing person when the facing person is directing the sightline of the facing person to the user, and not to match the sightline of the user with the facing person when the facing person is not directing the sightline of the facing person to the user. It is possible to evaluate in that these abilities are high when the pattern of the mutual sightline match and/or the second mutual sightline mismatch is dominant, for example.

The determination method according to the present disclosure can use any one or a plurality of configurations among the following plurality of configurations. In other words, any one or more configurations among the following plurality of configurations may be removed when the configurations are included in the embodiment, and may be added when the configurations are not included in the embodiment. By using such a configuration, it is possible to reproduce a sightline communication environment closer to real society, more reliably.

The determination method includes measuring the first mismatch time that the first mutual sightline mismatch is maintained.

The determination method includes calculating a degree of avoidance that the user avoids the mutual sightline match with the facing person based on the measured first mismatch time, and outputting the calculated degree of avoidance as a feedback.

The determination method includes outputting the measured first mismatch time as a feedback.

The determination method includes measuring the second mismatch time that the second mutual sightline mismatch is maintained.

The determination method includes calculating a degree of avoidance that the user avoids mutual sightline match with the facing person based on the measured second mismatch time, and outputting the calculated degree of avoidance as a feedback.

The determination method includes outputting the measured second mismatch time as a feedback.

The determination method includes measuring the third mismatch time that the third mutual sightline mismatch is maintained.

The determination method includes calculating a degree of sightline perseveration that is a condition in which the user is seeing at the facing person excessively based on the measured third mismatch time, and outputting the calculated degree of sightline perseveration as a feedback.

The determination method includes outputting the measured third mismatch time as a feedback.

The determination method includes outputting a different feedback for each of the mutual sightline match, the first mutual sightline mismatch, the second mutual sightline mismatch, and the third mutual sightline mismatch

When the user is facing at least the first facing person and the second facing person as the facing person, the determination method according to the present disclosure includes the following steps:
determining whether the sightline of the user is directed to the first facing person, and determining whether the sightline of the user is directed to the second facing person.
determining whether a sightline of the first facing person is directed to the user, and determining whether a sightline of the second facing person is directed to the user; and
outputting a feedback to the user based on results of the first determination and the second determination for the first facing person, and outputting a feedback to the user based on results of the first determination and the second determination for the second facing person.

The treating method, the training method, the evaluation method, and the monitoring method according to the present disclosure use the determination method according to the present disclosure.

With the treating method and the training method according to the present disclosure, it is possible to reproduce an environment of communication through a sightline which is close to real society easily and uniformly and to output a feedback, through the determination method, which can realize a treating method and a training method with high continuity and great effects. Effects of treatment with the treating method according to the present disclosure and effects of training with the training method according to the present disclosure mean expectation for improvement of at least any one of the following: the Liebowitz Social Anxiety Scale, the Social Phobia Inventory, the Patient Health Questionnaire-9, the Generalized Anxiety Disorder-7, the Health-Related QOL assessment (self-writing style) using the health economic Health-Related QOL Assessment Scale EuroQol 5 Dimension (EQ-5D), a feeling of anxiety about eye contact, a pleasure of eye contact, a degree of avoidance that the user avoids a mutual sightline match with the facing person based on the first mismatch time, a degree of avoidance that the user avoids a sightline match with the facing person based on the second mismatch time, and/or a degree of sightline perseveration based on the third mismatch time.

With the treating method and the training method according to the present disclosure, the user can adjust a difficulty level of a task and a determination, which can reduce anxiety and stress of the user.

With the treating method and the training method according to the present disclosure, it is possible to provide standardized treatment and training, regardless of expertise and proficiency of an executor (for example, a doctor, a psychological specialist (psychologist), a counselor, and a therapist), even in a situation where the executor is at a remote location or in a situation where the executor is absent. This can reduce variations in scores of the sightline communication ability and the social sightline control ability from use day to use day, which enables using these scores as quantitative indices. Namely, with the evaluating method and the monitoring method according to the present disclosure, it is possible to realize an evaluating method and a monitoring method which enable quantitatively evaluating the sightline communication ability through the determination method. The treating method according to the present disclosure also includes a treatment supporting method.

The social sightline control ability may also be recorded over time to monitor treatment or training effects. For example, by causing the same user to repeat training, the same user can confirm that his or her social sightline control ability has been improved since the user has become able to perform sightline match which the user was not able to perform before, the user has become able to increase the number of times that the sightline matches, and the like, in the same task. By performing training with an aim of improving the social sightline control ability, it is possible to expect improvement and enhancement of the sightline communication ability.

Examples of the social sightline transmission ability include “Power Gazing” indicating that the user himself or herself is in contact with another person with confidence, “Social Gazing” indicating that the user indicates an intention to have communication, and “Intimate Gazing” indicating that the user indicates an intention to be intimate with another person.

The training according to the present disclosure may be constituted by an arbitrary task which provides one teaching for directing or not directing a sightline to a facing person.

The tasks may be decided depending on the sightline communication ability to be improved. The task is not limited to a particular task as long as the task is configured for the purpose of improving and enhancing the ability, but it is preferable that viewpoints of Cognitive behavioral therapy are incorporated therein. By modifying a cognitively-and-behavioral biased pattern in the manner of Cognitive behavioral therapy, it is possible to obtain effects of attention shift training performed in Cognitive behavioral therapy, in addition to simply acquiring the social sightline control ability.

For example, patients with social anxiety disorder are in a state of inclining (biasing) their attention toward themselves (inside), and being unable to flexibly switch their attention. Therefore, by performing a task which provides a teaching for paying attention to recognition of facial expression or emotion of another person (for example, “Please look at the entire face of the partner. What kind of facial expression is it?” or “Please look at the entire face of the partner. How is the partner feeling!?”), it is possible to obtain an effect of facilitating the social sightline shift to the outside (another person or the like) in training for the social sightline shifting ability.

For example, patients with autism spectrum disorder are considered to have difficulties in “set sifting (cognitive flexibility)” and “central coherence (overall integrity)” which are cognitive tasks. Therefore, by performing a task which provides a teaching for looking at a part such as “Please look at both eyes” and a teaching for looking at the entire face such as “Please look at the entire face”, interweavingly, it is possible to obtain an effect of enhancing “central coherence (overall integrity)”, as well as “set shifting (cognitive flexibility)”, in training for the social sightline shifting ability.

It is also possible to obtain the social sightline transmission ability by performing a task including a teaching for causing the user to indicate that he or she is being in contact with another person with confidence (for example, “Please look at the partner's forehead and both eyes alternately.”), a teaching for causing the user to indicate his or her intention to have communication (for example, “Please look at the partner's both eyes and mouth alternately.”), or a teaching for causing the user to indicate his or her intention to be intimate with another person (for example, “Please look at the partner's eyes and chest alternately.”).

Conventionally, in Cognitive behavioral therapy, there has been formulated an auditory attention shift training method called “Attention Training Technique (ATT)”, which includes distinguishing a plurality of sounds from each other by listening. The training according to the present disclosure is very advantageous in that this training can embody attention shift training using the visual sense by combining sightline coincidence determination performed by the training system according to the present disclosure with tasks modified in the cognitive behavioral manner.

Namely, the training may include a first task in which the determination region P is set such that it includes a constituent part (for example, the eyes, the nose, the mouth, the forehead, and the like) of a social object to which the sightline is to be directed, and a second task in which the determination region P is set such that it includes a constituent unit encompassing the constituent part (for example, when the eyes are a constituent part, the constituent unit is the face, the entire body, or the like). The training may also include a third task in which the determination region P is set such that it includes the constituent part and the constituent unit alternately, and a fourth task which causes the user to estimate the facial expression and/or emotion of the social object in which the determination region P is set.

The training is performed by causing the user to execute one or more tasks. For example, the training may be performed by execution of a plurality of tasks which are successively presented. A plurality of trainings may be performed as a series of trainings, at daily or hourly intervals. A result of the training can be calculated as evaluation for each task or comprehensive evaluation for a plurality of tasks. Each task is a unit of sightline coincidence determinations which are successively performed, each sightline coincidence determination being performed for a predetermined time (for example, 20 seconds or 30 seconds), and is configured in such a way as to obtain at least one result of sightline coincidence determination.

The difficulty level of the training may be set according to the difficulty level of the determination and/or the difficulty level of the task. The difficulty of the determination may be adjusted by enlarging or reducing a determination region, a range of directions of the face, and a range from a center of the view image, and/or increasing or decreasing the maintaining time. The difficulty level of the task may be adjusted by selecting a scene which uses the determination device, the determination method, the training method, and the program. At this time, by monitoring a subjective or an objective stress of the user, the user can use it without feeling excessive stress. The objective stress of the user means stress evaluated by a person other than the user, and examples of this stress index include a social sightline control ability evaluated during training, a heart rate and a heart-rate variation index during training, a degree of opening of the pupils during training, a blinking frequency during training, and changes thereof. Examples of the heart-rate variation index include a low frequency component (LF), a high frequency component (HF) and/or the ratio therebetween (LF/HF). The stress exerted on the user may be determined based on generally-expected liability to exert stress. For example, it can be said that a task constituted by a scene in which the user asks a person having a fearful appearance about a direction provides a situation which is more liable to exert stress on the user than a task constituted by a scene in which the user asks a person having a gentle appearance about a direction.

The difficulty level of a task is changed, for example, by changing elements constituting the task. Examples of elements constituting a task include, for example, those shown in Table 1, in addition to a teaching for directing a sightline to a social object.

TABLE 1

| Constituent | |
|---|---|
| element | Concrete example |
| Object | Animal, personified animal (living being intermediate |
| | between animal and human), human (male, female) |
| Age | Baby, child, adult, elderly person |
| Number of | One or plural |
| persons | |
| Facial | Smiling face, angry face, expressionless face, good mood, |
| expression, | bad mood, gentle, fearful |
| impression | |
| Scene | Home, workplace (meeting, interview, speech, self- |
| | introduction), commercial facilities (convenience store, |
| | supermarket, restaurant), asking directions in strange lands |
| Distance | Close, far, getting closer, getting far away |
| to object | |

The present disclosure may be applied to a cognitive behavioral therapy. For example, the determination device 10, and the determination method and training method according to the present disclosure may be used as a part of a cognitive behavioral therapy.

As a method for presenting a task to the user, there are a method for presenting a task using a video content (a moving image and/or a still image), a method for presenting a task using a VR space, and a method for presenting a task in the real world. The difficulty level of a task may be changed by a selected presenting method.

In a case of presenting a task using an image, the difficulty level of a task may be changed by a form of the image (a moving image, a still image) and a type of the image (an Illustration, an animation, a photograph).

The training according to the present disclosure can decide and change the task in the manner described above. Therefore, it is possible to create a menu customized for each user, for example.

In a case of utilizing the training according to the present disclosure for treatment for a neuropsychiatric disorder, a time period of the training, a frequency of the training, and an execution time period per the single training are not particularly limited, and can be arbitrarily set by the user and/or the executor. Preferably, within a time period of 1 day to 1 year, the training is executed for 1 minute to 240 minutes per once, at a frequency of once per 90 days to 12 times per day. By performing the training for 1 minute only once per 90 days, it is possible to notice significance of sightline coincidence and/or eye contact with a social object, which raises expectation for effects of treatment. By intensively performing the training for 10 minutes once a day, 12 times in total, it is possible to raise expectation for effects of treatment. By performing the training at a slow pace of 20 minutes per once and once per 90 days, during 1 year, it is possible to raise expectation for effects of treatment. The execution time period per the single training may be arbitrarily set. However, in a case where the execution time period per the single training is less than 1 minute, there is a tendency to reduce the effect of treatment since the execution time period is too short. In a case where the execution time period per the single training exceeds 240 minutes, a load exerted on the user may be too large, which may reduce the effect of treatment. The frequency of execution of the training may be arbitrarily set. However, in a case of performing the training at a frequency lower than once per 90 days (for example, once per 120 days), intervals between the trainings is too long, which may reduce the effect of treatment. In a case of performing the training at a frequency higher than 12 times per day (for example, 20 times per day), a load exerted on the user may be too large, which may reduce the effect of treatment.

For example, in a case where the user visits a medical institution to undergo treatment, a time period of the raining, a frequency of the training, and an execution time period per the single training are not particularly limited, and can be arbitrarily set by the user and/or the executor. Preferably, the training is performed for 10 minutes to 40 minutes per once, 8 to 16 times in total, at a frequency of once or twice per week. This is because in consideration of the fact that the user visits a hospital about once a week in general, for example, assuming that the training for 20 minutes per once is performed once a week, it is considered that about 8 weeks are required to habituate the user thereto. In a case of performing treatment or the training outside a medical institution, for example, at home, a time period of the training, a frequency of the training, and an execution time period per the single training are not particularly limited, and can be arbitrarily set by the user. Preferably, the training for 10 minutes to 40 minutes per once is performed at a frequency of once a day or once a week. The total number of the trainings is not particularly limited, but it is preferable to perform the trainings in a range of 8 times to 30 times. It is also possible to use ambulatory treatment at a medical institution and home treatment in combination.

The training according to the present disclosure may also be applied to training of a person who has tended to deteriorate in sightline communication ability, or a person who may have a difficulty in having social life due to deterioration of his or her sightline communication ability, as well as a person having been diagnosed as having a neuropsychiatric disorder. In this case, similarly, a time period of the training, a frequency of training, and an execution time period per the single training are not particularly limited, and may be arbitrarily set by the user and/or the executor. The time period and the frequency are not particularly limited. Preferably, the training for 10 to 40 minutes per once is performed at a frequency of once a day or once a week.

The determination device 10 and the determination method may be configured in the manner described below.

The measurement unit 15 and the calculation unit 16 may be omitted. That is, it is possible to omit a step of “measuring the first mismatch time, or the second mismatch, or the third mismatch”, the step of “calculating the degree that the user avoids the mutual sightline match with the facing person based on the first mismatch time or the second mismatch time”, and the step of “calculating the degree of sightline perseveration based on the third mismatch time”. In this case, for example, the output unit 14 may be configured to output a feedback corresponding to the first sightline mismatch when the determination of the first sightline mismatch is made.

The determination device 10 may be provided with configurations corresponding to the first measurement unit, the second measurement unit, and the third measurement unit, instead of the measurement unit 15.

The determination device 10 may be provided with configurations corresponding to the first calculation unit, the second calculation unit, and the third calculation unit, instead of the calculation unit 16.

The configurations included in the determination device 10 may be located in the same device, or may be located in any of a plurality of device communicatively connected to one another. For example, the acquisition unit 11, the first determination unit 12, and the second determination unit 13 may be located in a first device, and the output unit 14 may be located in a second device that is communicatively connected to the first device.

In the flowcharts in FIGS. 10 to 13, step S43, step S48, step S51, and step S54 may be omitted.

The determination method according to the present disclosure may be executed by a computer. In other words, the present disclosure includes a program for causing a computer to execute the determination method, and a computer-readable storage medium that stores therein the program for causing a computer to execute the determination method.

Various embodiments according to the present disclosure have been described above in detail with reference to the drawings. Finally, various aspects according to the present disclosure will be explained.

A determination device according to a first aspect of the present disclosure includes:
an acquisition unit configured to acquire a sightline direction of a user and a sightline direction of at least one facing person facing the user;
a first determination unit configured to determine whether a sightline of the user is directed to the facing person based on the sightline direction of the user acquired by the acquisition unit;
a second determination unit configured to determine whether a sightline of the facing person is directed to the user based on the sightline direction of the facing person acquired by the acquisition unit; and
an output unit configured to output a feedback to the user and/or an executor based on a result of a determination of the first determination unit and a result of a determination of the second determination unit.

A determination device according to a second aspect of the present disclosure includes a first measurement unit configured to measure a first mismatch time that a first mutual sightline mismatch is maintained, the first mutual sightline mismatch being a condition in which the sightline of the user is not determined as being directed to the facing person but the sightline of the facing person is determined as being directed to the user.

A determination device according to a third aspect of the present disclosure includes a first calculation unit configured to calculate a degree of avoidance that the user avoids a mutual sightline match with the facing person based on the first mismatch time as measured by the first measurement unit, wherein
the output unit outputs the degree of avoidance calculated by the first calculation unit as a feedback.

In a determination device according to a fourth aspect of the present disclosure, the output unit outputs the first mismatch time as measured by the first measurement unit as a feedback.

A determination device according to a fifth aspect of the present disclosure includes a second measurement unit configured to measure a second mismatch time that a second mutual sightline mismatch is maintained, the second mutual sightline mismatch being a condition in which the sightline of the user is not determined as being directed to the facing person and the sightline of the facing person is not determined as being directed to the user.

A determination device according to a sixth aspect of the present disclosure includes
a second calculation unit configured to calculate a degree of avoidance that the user avoids a mutual sightline match with the facing person based on the second mismatch time as measured by the second measurement unit, wherein
the output unit outputs the degree of avoidance calculated by the second calculation unit as a feedback.

In a determination device according to a seventh aspect of the present disclosure, the output unit outputs the second mismatch time as measured by the second measurement unit as a feedback.

A determination device according to an eighth aspect of the present disclosure includes a third measurement unit configured to measure a third mismatch time that a third mutual sightline mismatch is maintained, the third mutual sightline mismatch being a condition in which the sightline direction of the user is determined as being directed to the facing person but the sightline of the facing person is not determined as being directed to the user.

A determination device according to a ninth aspect of the present disclosure includes a third calculation unit configured to calculate a degree of sightline perseveration that is a condition in which the user is seeing at the facing person excessively, based on the third mismatch time measured by the third measurement unit, wherein
the output unit outputs the degree of sightline perseveration calculated by the third calculation unit as a feedback.

In a determination device according to a tenth aspect of the present disclosure, the output unit outputs the third mismatch time measured by the third measurement unit as a feedback.

In a determination device according to a first of an eleventh aspect of the present disclosure,
when a condition in which the sightline of the user is determined as being directed to the facing person and the sightline of the facing person is determined as being directed to the user is defined as a mutual sightline match, a condition in which the sightline of the user is not determined as being directed to the facing person but the sightline of the facing person is determined as being directed to the user is defined as a first mutual sightline mismatch, a condition in which the sightline of the user is not determined as being directed to the facing person and the sightline of the facing person is not determined as being directed to the user is defined as a second mutual sightline mismatch, and a condition in which the sightline of the user is determined as being directed to the facing person but the sightline of the facing person is not determined as being directed to the user is defined as a third mutual sightline mismatch,
the output unit outputs a different feedback for each of the mutual sightline match, the first mutual sightline mismatch, the second mutual sightline mismatch, and the third mutual sightline mismatch.

In a determination device according to a second of the eleventh aspect of the present disclosure,
when a determination of a mutual sightline match is made based on a determination result indicating that the sightline of the user is directed to the facing person and that the sightline of the facing person is directed to the user; when a determination of a first mutual sightline mismatch is made based on a determination result indicating that the sightline of the user is not directed to the facing person but that the sightline of the facing person is directed to the user; when a determination of a second mutual sightline mismatch is made based on a determination result indicating that the sightline of the user is not directed to the facing person and the sightline of the facing person is not directed to the user; and when a determination of a third mutual sightline mismatch is made based on a determination result indicating that the sightline of the user is directed to the facing person but the sightline of the facing person is not directed to the user,
the output unit outputs a different feedback for each of the determination of the mutual sightline match, the determination of the first mutual sightline mismatch, the determination of the second mutual sightline mismatch, and the determination of the third mutual sightline mismatch.

In a determination device according to a twelfth aspect of the present disclosure,
when the user faces at least a first facing person and a second facing person as the facing person,
the first determination unit is configured to determine whether the sightline of the user is directed to the first facing person, and determine whether the sightline of the user is directed to the second facing person;
the second determination unit is configured to determine whether a sightline of the first facing person is directed to the user, and determine whether a sightline of the second facing person is directed to the user; and
the output unit is configured to output a feedback to the user based on determination results of the first determination unit and the second determination unit for the first facing person, and output a feedback to the user based on determination results of the first determination unit and the second determination unit for the second facing person.

A determination method according to a thirteenth aspect of the present disclosure includes:
acquiring a sightline direction of a user and a sightline direction of at least one facing person;
performing a first determination of determining whether a sightline of the user is directed to the facing person based on the acquired sightline direction of the user;
performing a second determination of determining whether a sightline of the facing person is directed to the user based on the acquired sightline direction of the facing person; and
outputting a feedback to the user and/or an executor based on a result of the first determination and a result of the second determination.

A determination method according to a fourteenth aspect of the present disclosure further includes measuring a first mismatch time that a first mutual sightline mismatch is maintained, the first mutual sightline mismatch being a condition in which the sightline of the user direction is not determined as being directed to the facing person but the sightline of the facing person is determined as being directed to the user.

A determination method according to a fifteenth aspect of the present disclosure further includes:
calculating a degree of avoidance that the user avoids the mutual sightline match with the facing person based on the measured first mismatch time; and
outputting the calculated degree of avoidance as a feedback.

A determination method according to a sixteenth aspect of the present disclosure includes outputting the measured first mismatch time as a feedback.

A determination method according to a seventeenth aspect of the present disclosure further includes measuring a second mismatch time that a second mutual sightline mismatch is maintained, the second mutual sightline mismatch being a condition in which the sightline of the user is not determined as being directed to the facing person and the sightline of the facing person is not determined as being directed to the user.

A determination method according to an eighteenth aspect of the present disclosure includes:
calculating a degree of avoidance that the user avoids mutual sightline match with the facing person based on the measured second mismatch time and
outputting the calculated degree of avoidance as a feedback.

A determination method according to a nineteenth aspect of the present disclosure further includes outputting the measured second mismatch time as a feedback.

A determination method according to a twentieth aspect of the present disclosure further includes measuring a third mismatch time that a third mutual sightline mismatch is maintained, the third mutual sightline mismatch being a condition in which the sightline of the user is determined as being directed to the facing person but the sightline of the facing person is not determined as being directed to the user.

A determination method according to a twenty-first aspect of the present disclosure further includes:
calculating a degree of sightline perseveration that is a condition in which the user is seeing at the facing person excessively based on the measured third mismatch time; and
outputting the calculated degree of sightline perseveration as a feedback.

A determination method according to a twenty-second aspect of the present disclosure further includes outputting the measured time length that the third mutual sightline mismatch is maintained, as a feedback.

A determination method according to a first of a twenty-third aspect of the present disclosure includes,
when a condition in which the sightline of the user is determined as being directed to the facing person and the sightline of the facing person is determined as being directed to the user is defined as a mutual sightline match, a condition in which the sightline of the user is not determined as being directed to the facing person but the sightline of the facing person is determined as being directed to the user is defined as a first mutual sightline mismatch, a condition in which the sightline of the user is not determined as being directed to the facing person and the sightline of the facing person is not determined as being directed to the user is defined as a second mutual sightline mismatch, and a condition in which the sightline of the user is determined as being directed to the facing person but the sightline of the facing person is not determined as being directed to the user is defined as a third mutual sightline mismatch,
outputting a different feedback for each of the mutual sightline match, the first mutual sightline mismatch, the second mutual sightline mismatch, and the third mutual sightline mismatch.

A determination method according to a second of a twenty-third aspect of the present disclosure includes,
when a determination of a mutual sightline match is made based on a determination result indicating that the sightline of the user is directed to the facing person and that the sightline of the facing person is directed to the user; when a determination of a first mutual sightline mismatch is made based on a determination result indicating that the sightline of the user is not directed to the facing person but that the sightline of the facing person is directed to the user; when a determination of a second mutual sightline mismatch is made based on a determination result indicating that the sightline of the user is not directed to the facing person and the sightline of the facing person is not directed to the user; and when a determination of a third mutual sightline mismatch is made based on a determination result indicating that the sightline of the user is directed to the facing person but the sightline of the facing person is not directed to the user,
outputting a different feedback for each of when the determination of the mutual sightline match is made based on the first determination and the second determination, when the determination of the first mutual sightline mismatch is made based on the first determination and the second determination, when the determination of the second mutual sightline mismatch is made based on the first determination and the second determination, and when the determination of the third mutual sightline mismatch is made based on the first determination and the second determination.

A determination method according to a twenty-fourth aspect of the present disclosure, the determination method includes:
when the user faces at least a first facing person and a second facing person as the facing person,
determining whether the sightline of the user is directed to the first facing person, and making a determination of whether the sightline of the user is directed to the second facing person;
determining whether a sightline of the first facing person is directed to the user, and making a determination of whether a sightline of the second facing person is directed to the user; and
outputting a feedback to the user based on results of the first determination and the second determination for the first facing person, and outputting a feedback to the user based on results of the first determination and the second determination for the second facing person.

A determination system according to a twenty-fifth aspect of the present disclosure includes:
the determination device according to any one of the first to twelfth aspects; and
a sightline direction acquisition device configured to detect a sightline direction of the user and a sightline direction of the facing person, and to output the detected sightline directions of the user and the facing person to the determination device.

A training method according to a twenty-sixth aspect of the present disclosure includes using the determination method according to any one of the thirteenth to the twenty-fourth aspects.

A treating method according to a twenty-seventh aspect of the present disclosure includes conducting a training using the training method according to the twenty-sixth aspect.

An evaluation method according to a twenty-eighth aspect of the present disclosure includes using the determination method according to any one of the thirteenth to the twenty-fourth aspects.

A monitoring method according to a twenty-ninth aspect of the present disclosure includes using the determination method according to any one of the thirteenth to the twenty-fourth aspects.

A diagnostic method according to a thirtieth aspect of the present disclosure includes using the determination method according to any one of the thirteenth to the twenty-fourth aspects.

A program according to a thirty-first aspect of the present disclosure is configured to cause a computer to execute the determination method according to any one of the thirteenth to the twenty-fourth aspects.

By using an appropriate combination of any of the embodiments and modifications, it is possible to achieve the advantageous effects corresponding thereto. In addition, any combinations of embodiments, combinations of examples, or combinations of embodiments and examples are possible, and combinations of features across different embodiments or examples are also possible.

Although the present disclosure has been fully described in connection with preferred embodiments with reference to the accompanying drawings, various changes and modifications thereof will also be apparent to those skilled in the art. Such changes and modifications are to be understood as falling within the scope of the present disclosure, as set forth in the appended claims.

### REFERENCE SIGNS LIST

1 determination system
10 determination device
11 acquisition unit
12 first determination unit
13 second determination unit
14 output unit
15 measurement unit
16 calculation unit
17 processor
18 storage unit
19 communication unit
20 sightline direction acquisition device
40 view image
100 facing person

1. A determination device, comprising:
an acquisition unit configured to acquire a sightline direction of a user and a sightline direction of at least one facing person facing the user;
a first determination unit configured to determine whether a sightline of the user is directed to the facing person based on the sightline direction of the user acquired by the acquisition unit;
a second determination unit configured to determine whether a sightline of the facing person is directed to the user based on the sightline direction of the facing person acquired by the acquisition unit; and
an output unit configured to output a feedback to the user and/or an executor based on a result of a determination of the first determination unit and a result of a determination of the second determination unit.

2. The determination device according to claim 1, comprising
a first measurement unit configured to measure a first mismatch time that a first mutual sightline mismatch is maintained, the first mutual sightline mismatch being a condition in which the sightline of the user is not determined as being directed to the facing person but the sightline of the facing person is determined as being directed to the user.

3. The determination device according to claim 2, comprising
a first calculation unit configured to calculate a degree of avoidance that the user avoids a mutual sightline match with the facing person based on the first mismatch time as measured by the first measurement unit, wherein
the output unit outputs the degree of avoidance calculated by the first calculation unit as a feedback.

4. The determination device according to claim 2, wherein
the output unit outputs the first mismatch time as measured by the first measurement unit as a feedback.

5. The determination device according to claim 1, comprising
a second measurement unit configured to measure a second mismatch time that a second mutual sightline mismatch is maintained, the second mutual sightline mismatch being a condition in which the sightline of the user is not determined as being directed to the facing person and the sightline of the facing person is not determined as being directed to the user.

6. The determination device according to claim 5, comprising
a second calculation unit configured to calculate a degree of avoidance that the user avoids a mutual sightline match with the facing person based on the second mismatch time as measured by the second measurement unit, wherein
the output unit outputs the degree of avoidance calculated by the second calculation unit as a feedback.

7. The determination device according to claim 5, wherein
the output unit outputs the second mismatch time as measured by the second measurement unit as a feedback.

8. The determination device according to claim 1, comprising
a third measurement unit configured to measure a third mismatch time that a third mutual sightline mismatch is maintained, the third mutual sightline mismatch being a condition in which the sightline direction of the user is determined as being directed to the facing person but the sightline of the facing person is not determined as being directed to the user.

9. The determination device according to claim 8, comprising
a third calculation unit configured to calculate a degree of sightline perseveration that is a condition in which the user is seeing at the facing person excessively, based on the third mismatch time measured by the third measurement unit, wherein
the output unit outputs the degree of sightline perseveration calculated by the third calculation unit as a feedback.

10. The determination device according to claim 8, wherein
the output unit outputs the third mismatch time measured by the third measurement unit as a feedback.

11. The determination device according to claim 1, wherein
when a condition in which the sightline of the user is determined as being directed to the facing person and the sightline of the facing person is determined as being directed to the user is defined as a mutual sightline match, a condition in which the sightline of the user is not determined as being directed to the facing person but the sightline of the facing person is determined as being directed to the user is defined as a first mutual sightline mismatch, a condition in which the sightline of the user is not determined as being directed to the facing person and the sightline of the facing person is not determined as being directed to the user is defined as a second mutual sightline mismatch, and a condition in which the sightline of the user is determined as being directed to the facing person but the sightline of the facing person is not determined as being directed to the user is defined as a third mutual sightline mismatch,
the output unit outputs a different feedback for each of the mutual sightline match, the first mutual sightline mismatch, the second mutual sightline mismatch, and the third mutual sightline mismatch.

12. The determination device according to claim 1, wherein
when the user faces at least a first facing person and a second facing person as the facing person,
the first determination unit is configured to determine whether the sightline of the user is directed to the first facing person, and determine whether the sightline of the user is directed to the second facing person;
the second determination unit is configured to determine whether a sightline of the first facing person is directed to the user, and determine whether a sightline of the second facing person is directed to the user; and
the output unit is configured to output a feedback to the user based on determination results of the first determination unit and the second determination unit for the first facing person, and output a feedback to the user based on determination results of the first determination unit and the second determination unit for the second facing person.

13. A determination method, comprising:
acquiring a sightline direction of a user and a sightline direction of at least one facing person;
performing a first determination of determining whether a sightline of the user is directed to the facing person based on the acquired sightline direction of the user;
performing a second determination of determining whether a sightline of the facing person is directed to the user based on the acquired sightline direction of the facing person; and
outputting a feedback to the user and/or an executor based on a result of the first determination and a result of the second determination.

14. The determination method according to claim 13, comprising
measuring a first mismatch time that a first mutual sightline mismatch is maintained, the first mutual sightline mismatch being a condition in which the sightline of the user direction is not determined as being directed to the facing person but the sightline of the facing person is determined as being directed to the user.

15. The determination method according to claim 14, comprising
calculating a degree of avoidance that the user avoids the mutual sightline match with the facing person based on the measured first mismatch time; and
outputting the calculated degree of avoidance as a feedback.

16. The determination method according to claim 14, comprising
outputting the measured first mismatch time as a feedback.

17. The determination method according to claim 13, comprising
measuring a second mismatch time that a second mutual sightline mismatch is maintained, the second mutual sightline mismatch being a condition in which the sightline of the user is not determined as being directed to the facing person and the sightline of the facing person is not determined as being directed to the user.

18. The determination method according to claim 17, comprising
calculating a degree of avoidance that the user avoids mutual sightline match with the facing person based on the measured second mismatch time, and
outputting the calculated degree of avoidance as a feedback.

19. The determination method according to claim 17, comprising
outputting the measured second mismatch time as a feedback.

20. The determination method according to claim 13, comprising
measuring a third mismatch time that a third mutual sightline mismatch is maintained, the third mutual sightline mismatch being a condition in which the sightline of the user is determined as being directed to the facing person but the sightline of the facing person is not determined as being directed to the user.

21. The determination method according to claim 20, comprising
calculating a degree of sightline perseveration that is a condition in which the user is seeing at the facing person excessively based on the measured third mismatch time; and
outputting the calculated degree of sightline perseveration as a feedback.

22. The determination method according to claim 20, comprising
outputting the measured third mismatch time as a feedback.

23. A determination method according to claim 13, comprising,
when a condition in which the sightline of the user is determined as being directed to the facing person and the sightline of the facing person is determined as being directed to the user is defined as a mutual sightline match, a condition in which the sightline of the user is not determined as being directed to the facing person but the sightline of the facing person is determined as being directed to the user is defined as a first mutual sightline mismatch, a condition in which the sightline of the user is not determined as being directed to the facing person and the sightline of the facing person is not determined as being directed to the user is defined as a second mutual sightline mismatch, and a condition in which the sightline of the user is determined as being directed to the facing person but the sightline of the facing person is not determined as being directed to the user is defined as a third mutual sightline mismatch,
outputting a different feedback for each of the mutual sightline match, the first mutual sightline mismatch, the second mutual sightline mismatch, and the third mutual sightline mismatch.

24. The determination method according to claim 13, comprising
when the user faces at least a first facing person and a second facing person as the facing person,
determining whether the sightline of the user is directed to the first facing person, and determining whether the sightline of the user is directed to the second facing person;
determining whether a sightline of the first facing person is directed to the user, and determining whether a sightline of the second facing person is directed to the user; and
outputting a feedback to the user based on results of the first determination and the second determination for the first facing person, and outputting a feedback to the user based on results of the first determination and the second determination for the second facing person.

25. A determination system, comprising:
the determination device according to claim 1; and
a sightline direction acquisition device configured to detect a sightline direction of the user and a sightline direction of the facing person, and to output the detected sightline directions of the user and the facing person to the determination device.

26. A training method, comprising
using the determination method according to claim 13.

27. A treating method for neuropsychiatric disorders, comprising
conducting a training using the training method according to claim 26.

28. An evaluation method of a sightline communication ability, comprising
using the determination method according to claim 13.

29. A monitoring method, comprising
using the determination method according to claim 13.

30. A diagnostic method for neuropsychiatric disorders, comprising using the determination method according to claim 13.

31. A non-transitory computer-readable storage medium storing program for causing a computer to execute the determination method according to claim 13.
